(19) United States
(12) Patent Application Publication
(10) Pub. No.: US 2016/0339141 A1
(43) Pub. Date: Nov. 24, 2016

(54) Hydrogel Matrix Having a Non-Uniform Distribution of Oxygen Containing Cells

(71) Applicant: AVENT, INC., Alpharetta, GA (US)

(72) Inventor: John P. Gann, Tucson, AZ (US)

(21) Appl. No.: 15/113,883

(22) PCT Filed: Jan. 30, 2015
(86) PCT No.: PCT/US2015/013663

§ 371 Date: Jul. 25, 2016

Related U.S. Application Data
(60) Provisional application No. 61/934,167, filed on Jan. 31, 2014.

(51) Int. Cl. A61L 15/60; A61L 15/44; A61L 15/18; A61Q 19/08; A61K 8/02; A61K 8/81; A61K 8/73; A61K 8/22; A61L 15/22; A61K 8/04

(52) U.S. Cl. CPC A61L 15/60; A61L 15/225; A61L 15/44; A61L 15/18; A61K 8/042; A61K 8/0241; A61K 8/8158; A61K 8/022; A61K 8/731; A61K 8/22; A61Q 19/08; A61L 2300/412; A61L 2300/11; A61K 2800/412; A61K 2800/546

(57) ABSTRACT

An oxygen coating composition comprising a hydrogel matrix and oxygen containing cells that are non-uniformly distributed in the matrix, wherein the oxygen containing cells include a superabsorbent polymer and an oxygen catalyst is provided. A method of making an oxygen coating composition is also provided. The method includes polymerizing a superabsorbent polymer and an oxygen catalyst to form a sheet; dehydrating the sheet; pulverizing the sheet to form a superabsorbent polymer/oxygen catalyst powder; and combining the powder with a film forming polymer to form a hydrogel matrix in which the powder is dispersed is also provided. The particular components and arrangement of the oxygen coating composition can improve the shelf life and stability of the coating composition, and the method by which the composition is made can be part of a continuous process rather than a batch type process.

### RELATED APPLICATIONS

The present application claims priority to U.S. Provisional Application Ser. No. 61/934,167, filed on Jan. 31, 2014, which is incorporated herein in its entirety by reference thereto.

### BACKGROUND OF THE INVENTION

The present invention relates to the provision of a coating that contains oxygen that can be applied to various substrates.

Lack of oxygen (i.e., hypoxia) is commonly experienced by people in their extremities as they get older due to poor blood circulation as well as by people affected by conditions such as diabetes. Studies have also shown below normal, low oxygen tension in the skin of elderly people. This often leads to poor skin health and an excessive presence of visible conditions such as wrinkles, dryness, and lower skin elasticity. Over the years, cosmetic manufacturers have introduced skin formulations with a large variety of ingredients such as emollients, exfoliators, moisturizers, etc., in an attempt to retard these age related effects and improve and maintain skin health.

In addition to alleviating symptoms related to the normal decrease in oxygen delivery to the skin, oxygen applied to wounds as, for example, a dressing containing oxygen, can speed healing. The delivery of oxygen to the skin and wounds for common use is a technological challenge, since oxygen is quite reactive and unstable. As such, it has been difficult to provide high concentrations of oxygen for at home use because of this instability. Oxygen has, however, been provided in the form of a peroxide and a peroxide decomposition catalyst per U.S. Patent Application Publication No. 2006/0121101 to Ladizinsky. This publication provides such a treatment for intact skin through the use of a dressing that is applied to an area of the skin. The dressing generally has a rupturable reservoir containing an aqueous hydrogen peroxide composition and a hydrogel matrix layer having a peroxide decomposition catalyst. Unfortunately, the catalytic decomposition of hydrogen peroxide to oxygen is quite rapid and so the dressing includes a layer that is impermeable to oxygen on the outside so that the oxygen is held against the skin for the maximum time possible. While this dressing is useful for small areas of the skin, it is unworkable for large areas or irregularly shaped areas of skin.

Alternatively, U.S. Pat. No. 5,736,582 to Devillez proposes the use of hydrogen peroxide in the place of benzoyl peroxide in skin treatment compositions that also contain solvents for hydrogen peroxide. This allows the hydrogen peroxide to stay below a level that will damage the skin and to stay in solution in greater concentrations. A solvent such as dimethyl isosorbide along with water is taught as being effective in its skin treatment composition. No peroxide decomposition catalyst is present. Unfortunately, no data on oxygen concentration or generation are given, nor is the time required for oxygen liberation. While this method appears to be an advance over non-oxygen containing compositions, the lack of data makes it difficult to make objective judgments on the overall effectiveness of this approach. Given the concentrations of peroxide, however, it is doubtful that significant volumes of oxygen were generated.

U.S. Pat. No. 7,160,553 to Gibbins, et al. proposes a matrix made from a polymer network and a non-gellable polysaccharide having oxygen for the treatment of compromised tissue. A closed cell foam is used to contain the dissolved oxygen and can also deliver other active agents.

U.S. Pat. No. 5,792,090 to Ladin proposes a wound dressing having an oxygen permeable layer in contact with the skin with an oxygen solution supply reservoir proximate the oxygen permeable layer. The reservoir is adapted to receive an aqueous liquid capable of supplying oxygen through chemical reaction. Preferably, the aqueous liquid contains hydrogen peroxide and the reservoir contains an immobilized solid hydrogen peroxide decomposition catalyst such as manganese dioxide. The catalyst in the dressing generates oxygen upon the addition of hydrogen peroxide.

Despite the development of the aforementioned dressings, matrices, and compositions, a need currently exists for an easy-to-use technology that can impart the ability to deliver oxygen to the skin attached to various types of substrates such as plastics, foams, non-wovens, and paper based products. It would also be desirable to have this technology in a form such that it is amenable for continuous manufacturing processes rather than batch type processes. Further, a need exists for an oxygen delivery product that is more stable and has a longer shelf-life than currently available oxygen delivery products and that can provide non-uniformly distributed and/or separated clusters of closed cells containing oxygen so that additional functionality can be imparted to the product if desired. In addition, a product that may be used in a manner similar to known products would be most readily accepted by the consumer.

### SUMMARY OF THE INVENTION

The problem discussed above has found a solution to a large degree in the present invention, which describes an oxygen coating composition that includes a hydrogel matrix, where the hydrogel matrix includes oxygen containing cells that are non-uniformly distributed throughout the matrix.

In accordance with one embodiment of the present invention, an oxygen coating composition is provided. The oxygen coating composition includes a hydrogel matrix and oxygen containing cells that are non-uniformly distributed in the matrix, where the oxygen containing cells include a superabsorbent polymer and an oxygen catalyst.

In one particular embodiment, the oxygen containing cells can include a superabsorbent polymerized in an amount ranging from about 60 wt. % to about 99 wt. % superabsorbent and between about 0.1 wt. % and about 20 wt. % percent oxygen catalyst on a dry-weight basis. In one particular embodiment, the oxygen containing cells can have an average particle size ranging from about 10 micrometers to about 300 micrometers.

In another embodiment, the superabsorbent polymer can be crosslinked. In still another embodiment, the superabsorbent polymer can include polyacrylamide, polyacrylic acid, sodium polyacrylate, polyethylvinyl acetate, polyurethane, polyethylene oxide, polyvinyl alcohol, polyvinyl pyrrolidone, polyvinyl pyridine, polyamine, polyvinyl morpholinone, an ethylene maleic anhydride copolymer, a polyvinyl ether, an isobutylene maleic anhydride copolymer, or a combination thereof. In an additional embodiment, the oxygen containing cells can further include a polysaccharide.

In yet another embodiment, the oxygen catalyst can include sodium carbonate, cupric chloride, ferric chloride, manganese oxide, silver oxide, sodium iodide, catalase, lactoperoxidase, or a combination thereof.

Further, the hydrogel matrix can include a film forming polymer, where the film forming polymer can be carboxymethyl cellulose, hydroxyethyl cellulose, polyvinyl alcohol, a carbomer, or a combination thereof.

In another embodiment, the composition can include a peroxide as a reactant for forming the oxygen in the oxygen containing cells.

In still another embodiment, that composition can be coated onto a substrate. The substrate can be a dressing (e.g., a wound dressing or bandage) or a personal care product. In yet another embodiment, the oxygen coating composition can be in the form of a fiber or a strand.

In accordance with another embodiment of the present invention, a method of making an oxygen coating composition is provided. The method includes polymerizing a superabsorbent polymer and an oxygen catalyst to form a sheet; dehydrating the sheet; pulverizing the sheet to form a superabsorbent polymer/oxygen catalyst powder; and combining the powder with a film forming polymer to form a hydrogel matrix in which the powder is dispersed.

In one particular embodiment, the superabsorbent polymer can be crosslinked. In another embodiment, the powder can have an average particle size ranging from about 10 micrometers to about 300 micrometers.

In still another embodiment, the method can include introducing a peroxide to the hydrogel matrix and activating the oxygen catalyst, wherein activation of the oxygen catalyst results in the formation of a non-uniform distribution of oxygen containing cells within the hydrogel matrix.

The method can further include the step of extruding the composition onto a substrate, wherein the film forming polymer is un-crosslinked. In another embodiment, the method can include the step of crosslinking the film forming polymer after extrusion of the composition onto the substrate. In still another embodiment, the composition can be extruded onto the substrate in the form of a pattern or as a continuous layer.

Other features and aspects of the present invention are set forth in greater detail below.

### BRIEF DESCRIPTION OF THE FIGURES

A full and enabling disclosure of the present invention, including the best mode thereof to one skilled in the art, is set forth more particularly in the remainder of the application, including reference to the accompanying figures, in which:

FIG. 1 is a cross-sectional view of a dressing comprising a substrate and an oxygen coating as contemplated by the present invention; and

FIG. 2 is a photograph of a top view of a dressing according to one embodiment of the present invention, where the oxygen coating is applied to the substrate in the form of a pattern.

Repeat use of reference characters in the present specification and drawings is intended to represent the same or analogous features or elements of the present invention.

### DETAILED DESCRIPTION

Reference will now be made in detail to one or more embodiments of the invention, examples of the invention, examples of which are illustrated in the drawings. Each example and embodiment is provided by way of explanation of the invention, and is not meant as a limitation of the present invention. For example, features illustrated or described as part of one embodiment may be used with another embodiment to yield still a further embodiment. It is intended that the invention include these and other modifications and variations as coming within the scope and spirit of the invention.

Generally speaking, in the process of making the hydrogel matrix with oxygen containing cells that forms the oxygen coating of the present invention, a superabsorbent polymer is synthesized and oxygen catalyst (e.g., sodium carbonate, cupric chloride, ferric chloride, manganese oxide, silver oxide, sodium iodide, catalase, lactoperoxidase, etc.) is added during polymerization, along with optional ingredients. The superabsorbent polymer and oxygen catalyst mixture thus produced is dried until it is dehydrated. The dehydrated superabsorbent and catalyst polymer mixture is then pulverized using a hammer mill or the like, into a coarse powder having an individual particle size ranging from about 10 micrometers (μm) to about 300 μm.

The superabsorbent and catalyst polymer mixture powder can be used in a number of applications. In one desired application, the powder is incorporated into a hydrogel matrix. A thickening agent or film forming polymer such as a carboxymethyl cellulose, hydroxyethyl cellulose, polyvinyl alcohol, a carbomer, etc. can also be used in the making of the hydrogel matrix. Desirably, the individual superabsorbent polymer/oxygen catalyst powder particles are appropriately dispersed and suspended in the hydrogel matrix. The hydrogel matrix may contain other excipients like humectants and/or plasticizers including glycerin, propylene glycol, polyethylene glycol (PEG), etc. as desired depending on the particular application in which the hydrogel matrix will be utilized.

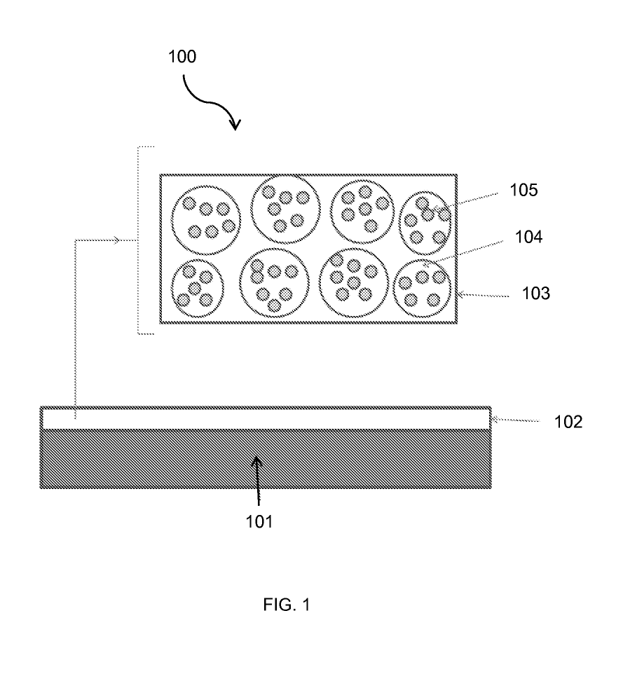

The hydrogel matrix may be coated onto a desired substrate in any suitable coating layer (e.g., a continuous layer) or pattern (e.g., a series of lines, dots, etc.) by extrusion, roll to roll coating, spin coating, or any other suitable known processes or may be extruded as a fiber or strands that may be formed into woven or nonwoven fabrics. As shown in FIG. 1, a wound dressing 100 is shown that incorporates a substrate 101 and an oxygen coating 102. The oxygen coating 102 includes a hydrogel matrix 103, in which is non-uniformly dispersed crosslinked superabsorbent polymer/oxygen catalyst powder particles 104 in which oxygen gas 105 is trapped.

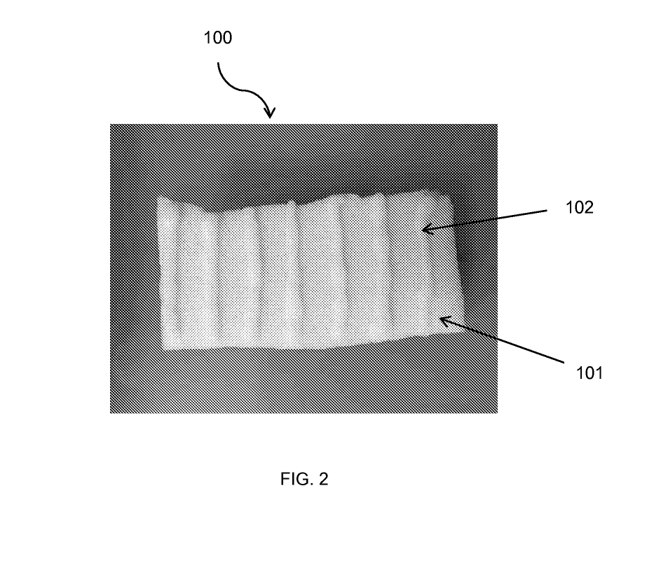

The incorporation of the crosslinked superabsorbent polymer/oxygen catalyst powder 104 into a free-flowing, un-crosslinked hydrogel matrix 103 allows for the formation of the oxygen coating 102 or patterned layer on a substrate as part of a continuous batch process. For instance, as shown in FIG. 2, the oxygen coating 102 can be applied to a substrate 101 in the form of a pattern of parallel lines or any other suitable pattern depending on the desired application of the dressing 100. Further, the hydrogel matrix 103 enhances the stability of the oxygen coating 102 by limiting the amount of diffusion of oxygen gas 105 out of the superabsorbent polymer/oxygen catalyst powder particles 104 by providing an additional barrier between the superabsorbent polymer and the environment. Such an arrangement can allow a substrate (e.g., a wound dressing) onto which the oxygen coating is applied to be stored at room temperature without loss of oxygen from the coating, thus eliminating the need to refrigerate the coated substrate. Although the hydrogel matrix component of the oxygen coating is un-crosslinked for application onto a substrate, it is to be understood that after application of the oxygen coating onto the substrate, the matrix can be crosslinked if desired.

Various embodiments of the present invention will now be described in more detail below.

### I. Superabsorbent Polymer/Oxygen Catalyst Powder

The superabsorbent polymer/oxygen catalyst powder component of the hydrogel matrix of the present invention can be formed by combining a superabsorbent polymer, a crosslinking agent, an oxygen catalyst, and other optional ingredients such as a polysaccharide, a hydration control agent, etc. into water.

Typically, a superabsorbent polymer is capable of absorbing at least about 10 times its weight in a 0.9 weight percent aqueous sodium chloride solution, and particularly is capable of absorbing more than about 20 times its weight in 0.9 weight percent aqueous sodium chloride solution. Superabsorbent polymers suitable for treatment or modification in accordance with the present invention are available from various commercial vendors, such as Dow Chemical Company located in Midland, Mich., USA, and Stockhausen Inc., Greensboro, N.C., USA. Other superabsorbent polymers suitable for treatment or modification in accordance with the present invention are described in U.S. Pat. No. 5,601,542 to Melius, et al.; U.S. Patent Application Publication No. 2001/0049514 to Dodge, et al.; and U.S. patent application Ser. No. 09/475,830 to Dodge, et al., each of which is hereby incorporated by reference in a manner consistent herewith.

The superabsorbent polymer can include, but is not limited to, an absorbent polymer such as polyacrylamide, polyacrylic acid, sodium polyacrylate, polyethylvinyl acetate, polyurethane, polyethylene oxide, polyvinyl alcohol, polyvinyl pyrrolidone, polyvinyl pyridine, polyamines, polyvinyl morpholinone, ethylene maleic anhydride copolymers, polyvinyl ethers, polymers and copolymers of vinyl sulfonic acid, isobutylene maleic anhydride copolymers, or a combination thereof. Other polymers that can be used include polylysine, polyethylene, polybuterate, polyether, silastic, silicone elastomer, rubber, nylon, vinyl, crosslinked dextran, or a combination thereof. If crosslinked dextran is used, it is preferred that the molecular weight of the dextran polymer is between 50,000 and 500,000. Additionally, the matrix material can be made from a combination of natural and synthetic polymers, or mixtures of synthetic polymers or mixtures of natural polymers. Exemplary natural polymers that can be used include, but are not limited to, hyaluronic acid and its derivatives, collagen, and starch derivatives (e.g., hydrolyzed acrylonitrile grafted starch, acrylic acid grafted starch, etc.). Other polymers contemplated by the present invention are citric acid based polymers, lactic acid and glycolic acid based polymers, poly(aspartates), poly(orthoesters), poly(phosphazenes), poly(anhydrides), poly(phosphoesters), and polyalkylene glycol based polymers, or a combination thereof. The superabsorbent materials of the present invention may be in any form suitable for use in absorbent structures, including, particles, fibers, flakes, spheres, and the like.

The superabsorbent polymer used to form the superabsorbent polymer/oxygen catalyst powder can be present in an amount ranging from about 0.25 wt. % and 20 wt. %, such as between about 0.5 wt. % and about 15 wt. %, such as between about 1 wt. % and about 10 wt. % based on the total weight of the powder solution prior to drying. On the other hand, after dehydration, the superabsorbent polymer can be present in an amount ranging from about 60 wt. % to about 99 wt. %, such as from about 70 wt. % to about 95 wt. %, such as from about 80 wt. % to about 90 wt. % based on the total weight of the superabsorbent polymer/oxygen catalyst powder on a dry-weight basis.

As mentioned above the superabsorbent polymer can be suitably lightly crosslinked to render the material substantially water-insoluble. Crosslinking may, for example, be by irradiation or by covalent, ionic, Van der Waals, or hydrogen bonding. One suitable crosslinking agent is N,N′-methylene-bisacrylamide, however other appropriate crosslinking agents such as bisacrylylycystamine and diallyltartar diamide may also be used. If N,N′-methylene-bisacrylamide or any other suitable crosslinking agent is used, it can be a component of the superabsorbent polymer/oxygen catalyst powder of the present invention in an amount ranging from about 0.005 wt. % to about 0.5% wt. %, such as from about 0.01 wt. % to about 0.25% wt. %, such as from about 0.025 wt. % to about 0.15 wt. % based on the total weight of the powder solution of the present invention prior to drying. On the other hand, after dehydration, the crosslinking agent can be present in an amount ranging from about 0.01 wt. % to about 4% wt. %, such as from about 0.05 wt. % to about 3% wt. %, such as from about 0.1 wt. % to about 2 wt. % based on the total weight of the superabsorbent polymer/oxygen catalyst powder on a dry-weight basis.

The superabsorbent polymer/oxygen catalyst powder can also include a polysaccharide, which can, in some embodiments, be a non-gellable polysaccharide. In one embodiment, a galactomannan macromolecule can be used in the wound treatment device. One example of such a macromolecule is guar gum. In another embodiment, the polysaccharide can be a cellulose or a cellulose derivative. For instance, methyl cellulose (MC), carboxymethyl cellulose (CMC), hydroxyethylcellulose (HEC), hydroxypropylmethyl cellulose (HPMC), hydroxypropyl cellulose (HPC), ethyl hydroxyethylcellulose (EHEC), or a combination thereof can be used in the present invention. Generally, the cellulose that can be used is modified so as to enhance the solubility of the cellulose in water. Another example of a suitable polysaccharide is dextrin. Dextrin is a non-crosslinked carbohydrate intermediate between starch and sugars that can be produced from starch through hydrolysis by dilute acids, distase, or dry heat, where the hydrolysis shortens the chains such that the solubility in water is increased. It has the general structure (C₆H₁₀O₅)ₓ, where x can be 6 or 7. In one particular embodiment, type III dextrin can be utilized. Other suitable examples of suitable polysaccharides include lucerne, fenugreek, honey locust bean gum, white clover bean gum, carob locust bean gum, xanthan gum, carrageenan, sodium alginate, chitin, chitosan, etc. The polysaccharide can be present in an amount ranging from about 0.005 wt. % and about 25% wt. %, such as between about 0.05 wt. % and about 10 wt. %, such as between about 0.1 wt. % and about 5 wt. %, based on the total weight of the superabsorbent polymer/oxygen catalyst powder solution prior to drying. On the other hand, after dehydration, the polysaccharide can be present in an amount ranging from about 0.1 wt. % to about 20 wt. %, such as from about 0.5 wt. % to about 15 wt. %, such as from about 1 wt. % to about 10 wt. % based on the total weight of the powder on a dry-weight basis.

As mentioned above, the powder present in the hydrogel matrix of the present invention also includes an oxygen catalyst. The catalyst may be sodium carbonate. However, other catalysts such as other alkali and alkali earth compounds may be used provided they are consistent with the product being biocompatible. In addition, more than one catalyst may be used. For instance, one catalyst may be derived from a group consisting of salts of alkali metals and alkali earth metals and the second catalyst may include, but are not limited to, organic and inorganic chemicals such as cupric chloride, ferric chloride, manganese oxide, silver oxide, sodium iodide and their equivalents. Other catalysts, include, but are not limited to enzymes such as lactoperoxidase and catalase. The catalyst can be present in an amount ranging from about 0.005 wt. % and about 5 wt. %, such as between about 0.01 wt. % and about 2.5 wt. %, such as between about 0.05 wt. % and about 1 wt. % based on the total weight of superabsorbent polymer/oxygen catalyst powder solution prior to drying. On the other hand, after dehydration, the catalyst can be present in an amount ranging from about 0.1 wt. % to about 20 wt. %, such as from about 0.5 wt. % to about 15 wt. %, such as from about 1 wt. % to about 10 wt. % based on the total weight of powder on a dry-weight basis.

Although not required, the biocompatible matrix may further include a hydration control agent. The hydration control agent may be an isopropyl alcohol such as isopropanol; however, ethanol, glycerol, butanol, and/or propylene glycol and combinations thereof may also be used. If present, the hydration control agent can be present in an amount ranging from about 0.05 wt. % to about 7.5% wt. %, such as from about 0.1 wt. % to about 5 wt. %, such as from about 0.5 wt. % to about 2.5 wt. % based on the total weight of the superabsorbent polymer/oxygen catalyst powder solution prior to drying. On the other hand, after dehydration, the hydration control agent can be present in an amount of less than 0.2 wt. %, such as less than about 0.1 wt. %, such as about 0 wt. % based on the total weight of powder on a dry-weight basis.

Ammonium persulfate and tetramethylethylenediamine (TEMED) may also be included in the superabsorbent polymer/oxygen catalyst powder solution of the present invention. The ammonium persulfate can be a component of the powder solution in an amount ranging from about 0.005 wt. % to about 0.5 wt. %, such as from about 0.01 wt. % to about 0.25 wt. %, such as from about 0.025 wt. % to about 0.1 wt. % based on the total weight of the superabsorbent polymer/oxygen catalyst powder solution prior to drying. On the other hand, after dehydration, the ammonium persulfate can be present in an amount of less than 0.2 wt. %, such as less than about 0.1 wt. %, such as about 0 wt. % based on the total weight of powder on a dry-weight basis. Additionally, the TEMED can be a component of the powder solution in an amount ranging from about 0.001 wt. % to about 0.5% wt. %, such as about 0.01 wt. % to about 0.25% w/w, such as about 0.025 wt. % to about 0.15% wt. % based on the total weight of the superabsorbent polymer/oxygen catalyst powder solution prior to drying. On the other hand, after dehydration, the TEMED can be present in an amount of less than 0.2 wt. %, such as less than about 0.1 wt. %, such as about 0 wt. % based on the total weight of powder on a dry-weight basis.

To form the solution from which the superabsorbent polymer/oxygen catalyst powder is formed, water or any other suitable aqueous solution can be added in an amount ranging from about 80 wt. % to about 99 wt. %, such as from about 85 wt. % to about 98 wt. %, such as from about 90 wt. % to about 95 wt. % based on the total weight of the superabsorbent polymer/oxygen catalyst powder solution prior to drying. On the other hand, after dehydration, water can be present in an amount of less than about 0.2 wt. %, such as less than about 0.1 wt. %, such as about 0 wt. % based on the total weight of the powder on a dry-weight basis.

In the process of making the oxygen coating composition disclosed herein, the superabsorbent polymer (e.g., polyacrylamide) is synthesized using established procedures where the crosslinking agent can be added. During the polymerization process, the oxygen catalyst is added (e.g., sodium carbonate, cupric chloride, ferric chloride, manganese oxide, silver oxide, sodium iodide, catalase, lactoperoxidase, etc.), although the oxygen catalyst is not believed to take part in the polymerization reaction that produces the superabsorbent polymer. The resulting superabsorbent polymer and oxygen catalyst solution thus produced is then dried until it is sufficiently dehydrated. The mixture may have between 60 and 99 weight percent superabsorbent and between about 0.1 wt. % and about 20 wt. % oxygen catalyst on a water-free basis, such as between about 80 wt. % and 90 wt. % superabsorbent polymer and between about 1 wt. % and about 10 wt. % oxygen catalyst on a dry weight or water-free basis. By “water-free” is meant the condition of the mixture after dehydrating or drying down to a moisture loss of between 60% and 80%. The dehydrated superabsorbent polymer and oxygen catalyst polymer mixture is then pulverized using a hammer mill or the like, into a coarse powder having an individual average particle size ranging from about 10 micrometers to about 300 micrometers, such as from about 25 micrometers to about 250 micrometers, such as from about 50 micrometers to about 200 micrometers.

### II. Hydrogel Matrix Formulation

The resulting powder formed from the superabsorbent polymer and oxygen catalyst solution discussed above can be used in a number of applications. In one application, the resulting powder can be incorporated into a hydrogel matrix formulation that can also include hydrogen peroxide or any other suitable reactant to form an oxygen coating composition, where the superabsorbent polymer/oxygen catalyst powder can be present as a non-uniform discontinuous phase dispersed within an amorphous hydrogel matrix material formed from, for example, a film forming polymer. The powder can be present in the hydrogel matrix formulation in an amount ranging from about 0.25 wt. % to about 20 wt. %, such as from about 0.5 wt. % to about 15 wt. %, such as from about 1 wt. % to about 10 wt. % based on the total weight of the hydrogel matrix formulation prior to drying. Meanwhile, after drying the formulation to a desired moisture content, the superabsorbent polymer/oxygen catalyst powder can be present in the resulting oxygen coating composition in an amount ranging from about 5 wt. % to about 95 wt. %, such as from about 20 wt. % to about 80 wt. %, such as from about 40 wt. % to about 60 wt. %.

As discussed above, the hydrogel matrix formulation can include any suitable film forming polymer or thickening agent such as a carboxymethyl cellulose, hydroxyethyl cellulose, polyvinyl alcohol, a carbomer, any other suitable synthetic hydrophilic film former, or combinations thereof. The film forming polymer can be present in the oxygen coating composition in an amount ranging from about 0.1 wt. % to about 20 wt. %, such as from about 0.25 wt. % to about 15 wt. %, such as from about 0.5 wt. % to about 10 wt. % based on the total weight of hydrogel matrix formulation prior to drying. Meanwhile, after drying the formulation to a desired moisture content, the film forming polymer can be present in the resulting oxygen coating composition in an amount ranging from about 1 wt. % to about 35 wt. %, such as from about 1.5 wt. % to about 30 wt. %, such as from about 2 wt. %

to about 25 wt. %.

The hydrogel matrix formulation can also contain other excipients like humectants and/or plasticizers. The plasticizer can be glycerol/glycerin, propylene glycol, polyethylene glycol (PEG), butanol, or any other suitable plasticizer, and combinations. The plasticizer can be present in an amount ranging from about 1 wt. % and about 50 wt. %, such as between about 2.5 wt. % and about 40 wt. %, such as between about 5 wt. % and 30 wt. % based on the total weight of the hydrogel matrix formulation prior to drying. Meanwhile, after drying the formulation to a desired moisture content, the plasticizer can be present in the resulting oxygen coating composition in an amount ranging from about 10 wt. % to about 60 wt. %, such as from about 15 wt. % to about 55 wt. %, such as from about 20 wt. % to about 50 wt. %.

Meanwhile, water can be present in the hydrogel matrix formulation in an amount ranging from about 40 wt. % to about 95 wt. %, such as from about 45 wt. % to about 90 wt. %, such as from about 50 wt. % to about 85 wt. % based on the total weight of the hydrogel matrix formulation prior to drying. Meanwhile, after drying the formulation to a desired moisture content, water can be present in the resulting oxygen coating composition in an amount ranging from about 1 wt. % to about 35 wt. %, such as from about 2.5 wt. % to about 30 wt. %, such as from about 5 wt. % to about 25 wt. %.

Further, if added to the hydrogel matrix formulation before coating the formulation onto a substrate to form an oxygen coating composition on the substrate, a peroxide (e.g., hydrogen peroxide, ammonium peroxide, sodium peroxide, urea peroxide, calcium peroxide, or a combination thereof) or any other suitable reactant can be added to the hydrogel matrix formulation to react with the oxygen catalyst in the powder to form cells containing oxygen trapped inside the superabsorbent polymer component of the powder. If added to the formulation, the peroxide or other suitable reactant can be present in an amount ranging from about 0.1 wt. % to about 15 wt. %, such as from about 0.25 wt. % to about 10 wt. %, such as from about 0.5 wt. % to about 5 wt. % based on the total weight of the hydrogel matrix formulation prior to drying. Meanwhile, after drying the formulation to a desired moisture content, the peroxide or other suitable reactant can be present in the resulting oxygen coating composition in an amount ranging from about 0.01 wt. % to about 2 wt. %, such as from about 0.05 wt. % to about 1.5 wt. %, such as from about 0.1 wt. % to about 1 wt. %.

The hydrogel matrix mixture can be prepared by combining the film forming polymer, superabsorbent/catalyst powder, plasticizer, and any other components to form a slurry. Meanwhile, the peroxide may be diluted with water and then blended with the slurry before extrusion. Alternatively and as discussed below, the peroxide may be omitted from the formulation and sprayed onto the hydrogel matrix formulation after extrusion of the formulation onto a substrate, or the substrate coated with the hydrogel matrix formulation may be dipped into a hydrogen peroxide bath.

### III. Application of Hydrogel Matrix to a Substrate

As discussed above, after the hydrogel matrix formulation components are combined, the hydrogel matrix formulation may then be coated onto the desired substrate (e.g., polyurethane, gauze, or nonwoven material or any other suitable wound dressing) by extrusion, roll to roll coating, spin coating, or any other suitable known process in a free-flowing, un-crosslinked state, after which the formulation can optionally be crosslinked as discussed in more detail below to stabilize the hydrogel matrix on the substrate. The hydrogel matrix formulation can be extruded onto a substrate or coated using standard methods like a slot coater or Meyer rod.

As discussed above, the hydrogel matrix may be exposed to hydrogen peroxide or other suitable reactant by, for example, blending or compounding the hydrogen peroxide or other suitable reactant directly into the hydrogel matrix formulation. In the alternative, if the hydrogel matrix formulation is applied to a substrate prior to addition of hydrogen peroxide, the hydrogel matrix-coated substrate may be dipped into a hydrogen peroxide solution or the coated substrate may be sprayed with hydrogen peroxide.

If necessary, the peroxide-containing or coated hydrogel matrix formulation may be activated after the hydrogel matrix formulation is applied to the substrate to form an oxygen coating on the substrate. Such activation can be carried about by applying heat (e.g., heating the substrate for a time frame of from about 5 minutes to about 5 hours at a temperature of from about 30° C. to about 70° C., such as for about 2 hours at about 55° C.) to accelerate the breakdown of hydrogen peroxide into oxygen gas by the action of the oxygen catalyst trapped in the powder dispersed within the resulting hydrogel matrix. This expanded or “foamed” oxygen coating typically shows good adhesion to various substrates. The adhesion may be tailored to specific substrates by scrupulous choice of thickeners and excipients.

Unlike oxygen containing structures in which the oxygen containing cells are uniformly distributed, the hydrogel matrix containing the superabsorbent and oxygen catalyst powder of the present invention may provide separation between clusters of closed cells containing gaseous oxygen within a unitary structure (e.g., the hydrogel matrix). The reason such separation is important is that it allows additional functionalities to be included or accentuated. For example, a uniform, distributed through-out, arrangement (as a coating for example) is believed to be limited in wicking watery exudate away from a wound because of blocking or impeding of flow by other oxygen containing cells. This phenomenon is known in the art of producing personal care products containing superabsorbent as “gel blocking”.

In the non-uniform oxygen coating of the present invention, the parts of the hydrogel matrix not containing oxygen cells can more easily conduct water between the oxygen cell clusters, allowing for enhanced wicking. As another example, if there is ‘synergy’ or attraction between the hydrogel matrix and a ‘porous’ or mesh, gauze or fiber substrate, the matrix could selectively absorb into or onto the substrate while the catalyst powder could become concentrated in the void areas presented by the substrate proper. Again, the non-uniform distribution of oxygen containing cells within the hydrogel matrix can provide improved wicking and/or oxygen delivery to the wound.

If the film forming polymer component of the hydrogel matrix formulation is crosslinked after it has been applied to the substrate in order to minimize dissolution of the resulting coating should it be used, for example, in a wound with high amounts of exudate. The crosslinking method may be tailored to the individual chemistry of the film forming polymer. For example, one way to crosslink carboxymethyl cellulose is by exposure to trivalent cations by use of soluble salts of Fe³⁺, Al³⁺, and Cr³⁺. These salts can be dissolved and then sprayed onto the coating after it has been applied to the substrate, either before or after the oxygen foaming reaction has occurred.

Another way to achieve crosslinking in the coating is to incorporate low concentrations of the trivalent salts into the coating formulation as it is compounded. The subsequent water loss that occurs due to the heating that is needed to drive the foaming reaction will act to further crosslink the coating. Another possibility would involve the addition of gelatin or chitosan into the coating and then crosslinking these materials with genipin (a known biocompatible crosslinking agent). The genipin could either be sprayed on the coating after it is applied, or batch blended into the coating formulation and then activated during the heating step to induce crosslinking.

Another example of crosslinking would be the combination of polyvinyl alcohol in the film with sodium borate used as the crosslinker. Other methods of crosslinking such as UV irradiation and e-beam may be used as well. Alternatively, the hydrogel matrix may remain free-flowing and applied to a wound or substrate as a liquid.

Substrates that may be coated with the disclosed hydrogel matrix formulation include bandages, dressings, gauze, foam, etc. that may be used in wound care and nonwoven pads that may be used in personal care items like diapers and feminine hygiene products. The hydrogel matrix formulation can be extruded or coated onto such substrates as uniform layer or in any suitable pattern, such as the parallel lines shown in FIG. 2. Generally, the resulting oxygen coating composition can have a thickness of from about 0.025 millimeters to about 25 millimeters, such as from about 0.05 millimeters to about 20 millimeters, such as from about 0.1 millimeters to about 10 millimeters. Ultimately, after the oxygen coating composition is formed and after any drying of the hydrogel matrix formulation, the individual powder particles are appropriately dispersed and suspended in the hydrogel matrix, and the resulting oxygen coating composition can contain between about 5 wt. % to about 95 wt. % of superabsorbent material, based on the total weight of the fiber, the superabsorbent material, and/or any other components on a dry weight basis. Optionally, the wt. % of the superabsorbent material in the oxygen coating composition may be from about 20 wt. % to about 80 wt. %, such as from about 40 wt. % to about 60 wt. % on a dry-weight basis. In an alternative embodiment, the hydrogel matrix formulation may also be extruded into strands or fibers that may be formed into woven and nonwoven fabrics.

The present invention may be better understood with reference to the following example.

### EXAMPLE

The ability to form a two-layer structure including a first substrate layer and a second oxygen coating layer containing oxygen trapped within a superabsorbent polymer that is embedded within a hydrogel matrix is demonstrated. It is to be understood that the wt. % values given in Tables 1 and 2 are from one example only and that amounts for the ingredients can vary and remain within the spirit of the disclosure and other ingredients that provide equivalent results may be used.

### Step 1—Preparation of Superabsorbent and Oxygen Catalyst Powder

TABLE 1
Preparation of Superabsorbent Polymer and Catalyst Powder
for Use in Hydrogel Matrix O₂ Coating

| | | | | Wt. % |
|---|---|---|---|---|
| | | Example | | (Dehydrated, |
| | | (800 g | Order of | Post |
| Ingredient | Wt. % | batch) | Addition | Polymerization) |
| Water | 92.28 | 738.15 | 1 | 0 |
| Bis-Acrylamide | 0.07 | 0.56 | 2 | 1.07 |
| Acrylamide | 5.74 | 45.92 | 3 | 87.5 |
| Sodium Carbonate | 0.18 | 1.44 | 4 | 2.74 |
| Guar Gum | 0.57 | 4.56 | 5 | 8.69 |
| Isopropanol (IPA) | 1.03 | 8.24 | 5 | 0 |
| TEMED | 0.05 | 0.4 | 6 | 0 |
| Ammonium | 0.08 | 0.64 | 7 | 0 |
| Persulfate | | | | |

Note:
The guar gum and IPA were combined then added.

Once all of the above ingredients were combined in the amounts and order shown in Table 1, the resulting mixture was poured into shallow vessels and allowed to polymerize overnight. The resulting polymerized “sheets” were then dehydrated at an elevated temperature about 55° C. for 12 hours or until dehydrated. It can be determined that the dehydrated or “water-free” post-polymerization stage is sufficient when the material can be pulverized using a hammer mill into a fine powder, in this example resulting in a powder having a particle size of about 120 μm. The material was then stored as a dry powder and used as needed. This “catalyst gel powder” or “superabsorbent/catalyst powder” was then used as an ingredient in the hydrogel matrix made as described in step 2 below.

### Step 2—Preparation of the Hydrogel Matrix

The following Table 2 gives desirable ranges for each component as well as the function of each and an example of the desired weight percent of each component in the final hydrogel matrix formulation.

TABLE 2
Hydrogel Matrix Formulation

| | | Desired | Post Drying | |
|---|---|---|---|---|
| | Wt. | Ranges | Ranges | |
| Ingredient | % | (Wt. %) | (Wt. %) | Function |
| Water | 77 | 45-93% | 5-25% | Solubilizing agent for |
| | | | | CMC, blending |
| | | | | solvent |
| Hydrogen | 3 | 0.5-5% | 0.1-1% | Oxygen source part 1 |
| Peroxide | | | | |
| Carboxymethyl | 2 | 0.5-10% | 2-25% | Thickening agent, |
| Cellulose | | | | binding agent, |
| (CMC) | | | | film former |
| Glycerin | 15 | 5-30% | 20-50% | Dispersing agent for |
| | | | | catalyst gel powder |
| Superabsorbent | 3 | 1-10% | 20-80% | Oxygen source part 2, |
| Catalyst | | | | O₂ gas reservoir |
| Powder | | | | |

The hydrogel matrix mixture was prepared by combining the CMC, superabsorbent/catalyst powder (from step 1), and glycerin into a slurry. The hydrogen peroxide was then diluted with water and blended with the slurry before extrusion. As discussed above, it is also to be understood, however, that the hydrogen peroxide may be omitted from the mixture and sprayed onto the hydrogel matrix mixture after extrusion, or the coated structure may be dipped into a hydrogen peroxide bath.

The hydrogel matrix mixture was then extruded onto a substrate. Then, an oxygen (O₂) foaming reaction was initiated by heating the substrate at a sufficient temperature for a sufficient time (e.g., at about 55° C. for about 2 hours). This heating step is dependent upon the type of oxygen catalyst used, however, and so may be varied accordingly.

These and other modifications and variations of the present invention may be practiced by those of ordinary skill in the art, without departing from the spirit and scope of the present invention. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

1. An oxygen coating composition comprising a hydrogel matrix and oxygen containing cells that are non-uniformly distributed in the matrix, wherein the oxygen containing cells include a superabsorbent polymer and an oxygen catalyst.

2. The composition of claim 1, wherein the oxygen containing cells comprise a superabsorbent polymerized in an amount ranging from about 60 wt. % to about 99 wt. % superabsorbent and between about 0.1 wt. % and about 20 wt. % percent oxygen catalyst on a dry-weight basis.

3. The composition of claim 1, wherein the superabsorbent polymer is crosslinked.

4. The composition of claim 1, wherein the superabsorbent polymer comprises polyacrylamide, polyacrylic acid, sodium polyacrylate, polyethylvinyl acetate, polyurethane, polyethylene oxide, polyvinyl alcohol, polyvinyl pyrrolidone, polyvinyl pyridine, polyamine, polyvinyl morpholinone, an ethylene maleic anhydride copolymer, a polyvinyl ether, an isobutylene maleic anhydride copolymer, or a combination thereof.

5. The composition of claim 4, wherein the oxygen containing cells further comprise a polysaccharide.

6. The composition of claim 1, wherein the oxygen catalyst comprises sodium carbonate, cupric chloride, ferric chloride, manganese oxide, silver oxide, sodium iodide, catalase, lactoperoxidase, or a combination thereof.

7. The composition of claim 1, wherein the oxygen containing cells have an average particle size ranging from about 10 micrometers to about 300 micrometers.

8. The composition of claim 1, wherein the hydrogel matrix comprises a film forming polymer.

9. The composition of claim 8, wherein the film forming polymer comprises carboxymethyl cellulose, hydroxyethyl cellulose, polyvinyl alcohol, a carbomer, or a combination thereof.

10. The composition of claim 1, further comprising a peroxide.

11. A substrate coated with the composition of claim 1.

12. The substrate of claim 11, wherein the substrate comprises a dressing or a personal care product.

13. A strand or fiber comprising the composition of claim 1.

14. A method of making an oxygen coating composition comprising:
polymerizing a superabsorbent polymer and an oxygen catalyst to form a sheet;
dehydrating the sheet;
pulverizing the sheet to form a superabsorbent polymer/oxygen catalyst powder; and
combining the powder with a film forming polymer to form a hydrogel matrix in which the powder is dispersed.

15. The method of claim 14, wherein the superabsorbent polymer is crosslinked.

16. The method of claim 14, wherein the powder has an average particle size ranging from about 10 micrometers to about 300 micrometers.

17. The method of claim 14, further comprising introducing a peroxide to the hydrogel matrix and activating the oxygen catalyst, wherein activation of the oxygen catalyst results in the formation of a non-uniform distribution of oxygen containing cells within the hydrogel matrix.

18. The method of claim 14, further comprising extruding the composition onto a substrate, wherein the film forming polymer is un-crosslinked.

19. The method of claim 18, further comprising crosslinking the film forming polymer after extrusion of the composition onto the substrate.

20. The method of claim 18, wherein the composition is extruded onto the substrate in the form of a pattern or as a continuous layer.
